(19) United States
(12) Patent Application Publication
(10) Pub. No.: US 2018/0221530 A1
(43) Pub. Date: Aug. 9, 2018

(54) DEODORIZATION OF RECLAIMED STEAM FROM WASTE TREATMENT AUTOCLAVE

(71) Applicant: Sterilis, LLC, Boxborough, MA (US)

(72) Inventors: Eric Muhlanger, Arlington, MA (US); Daniel Boutilier, Pascoag, RI (US); Nathan Palmer, Allston, MA (US); Kostyantyn Barsukov, Brookline, MA (US); Robert Walsh, Nashua, NH (US); Duane Delfosse, Windham, NH (US)

(21) Appl. No.: 15/889,850

(22) Filed: Feb. 6, 2018

Related U.S. Application Data
(60) Provisional application No. 62/456,250, filed on Feb. 8, 2017.

(51) Int. Cl. A61L 11/00; B01D 53/14; B09B 3/00

(52) U.S. Cl. CPC A61L 11/00; B01D 2252/205; B09B 3/0075; B01D 53/1493

(57) ABSTRACT

Methods and apparatus for deodorization of steam from a waste treatment autoclave. A system includes an autoclave for sterilization of waste, the autoclave being fluidly linked or coupled to a bottom portion of a condensate tank to enable flow of steam from the autoclave to the condensate tank, and a deodorization tank containing a liquid deodorant. An upper portion of the condensate tank is fluidly linked or coupled to a bottom portion of the deodorization tank to enable steam/air from the condensate tank to bubble through the liquid deodorant and exit from a vent located on a top of the deodorization tank.

### CROSS-REFERENCE TO RELATED APPLICATIONS

This application claims the benefit of U.S. Provisional Application Ser. No. 62/456,250 filed Feb. 8, 2017, the teachings of which are incorporated herein by reference.

### BACKGROUND OF THE INVENTION

The present invention generally relates to deodorization, and more specifically to deodorization of reclaimed steam from a waste treatment autoclave.

In general, autoclaves that operate under steam pressure at high temperature are widely used for sterilization and decontamination of materials. The combination of steam and heat under pressure provide an excellent means of destroying contamination, such as microorganisms. A wide variety of materials introduced into the autoclave include various utensils, glassware, plastics, agar, media, and so forth. Heating the products placed in the autoclave can result in the release of odors that are not always pleasant, and upon opening the autoclave, the offensive emissions are usually detected by everyone that are in close proximity.

### SUMMARY OF THE INVENTION

Methods and apparatus for deodorization of reclaimed steam from a waste treatment autoclave.

According to a general aspect of the present invention there is featured a deodorizing system for an autoclave that uses a liquid filled condensing output tank (waste tank) and a deodorizing tank such that the output of the autoclave bubbles through the condensation tank and the output of the condensation tank bubbles through the deodorizer in the deodorizer tank.

According to a further aspect, such a system of the present invention includes a spraying device to distribute a deodorant onto the solid waste output from the autoclave.

According to further aspects, such a system of the present invention includes an overflow system that permits excess condensate to be drained to a facility drain or suitable device.

In embodiments, condensate from an autoclave is collected in a first reservoir. A conduit links a top portion of the first reservoir to a bottom portion of a second reservoir containing one or more deodorants. Air and expelled steam flow through the conduit from the first reservoir through one or more deodorants in the second reservoir and then expelled to the outside of the second reservoir.

In further embodiments, such a system features an autoclave for sterilization of waste, the autoclave fluidly linked to a bottom portion of a condensate tank to enable flow of steam from the autoclave to the condensate tank, and a deodorization tank containing a liquid deodorant, an upper portion of the condensate tank fluidly linked to a bottom portion of the deodorization tank to enable steam from the condensate tank to bubble through the liquid deodorant and exit from a vent located on a top of the deodorization tank.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The invention will be more fully understood by reference to the detailed description, in conjunction with the following figures, wherein:

FIG. 1 is a block diagram of an exemplary deodorization system.

FIG. 2 is a block diagram of an exemplary overflow plumbing system adapted to the deodorization system of FIG. 1.

FIG. 3 is a flow diagram.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

The present invention provides methods and apparatus for deodorization of reclaimed steam from a waste treatment autoclave.

The present invention will be described in the context of autoclave sterilization of medical waste. However, it should be appreciated that the system and method described herein may be used or adapted to handle deodorization of multiple types of waste that passes through an autoclave.

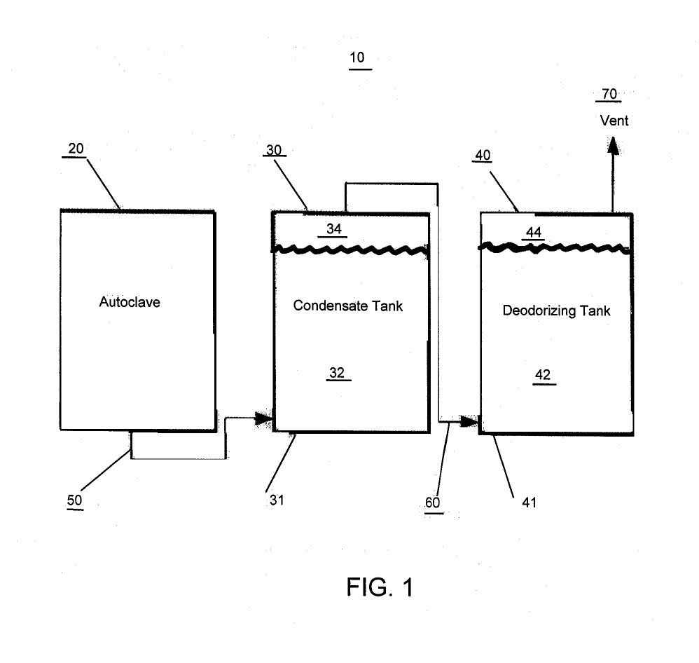

As shown in FIG. 1, an exemplary system 10 for deodorization of reclaimed steam from a waste treatment autoclave includes an autoclave 20 for sterilization of waste, a condensate tank 30 and a deodorization tank 40. In one example, the autoclave 20 is manufactured from stainless steel while the condensate tank 30 and deodorization tank 40 are manufactured from plastics. This shall not be limiting as the condensate tank and the deodorization tank can be made of any of number of materials known in the arts and otherwise appropriate for the intended use. For example, one or both of the tanks can be constructed from a metal such as stainless steel.

In one specific embodiment, the system 10 can optionally further include a spraying device (not shown) to distribute an atomized deodorant directly onto the sterilized solid waste output from the autoclave 20. Such a spraying device is any of a number of devices known in the art and appropriate for the intended use. Such a spraying device can be fluidly coupled to the deodorizing tank 40 or another source of deodorizing fluid/

A fluid line 50 links a bottom of the autoclave 20 with a lower portion 31 of the condensate tank 30. In addition, a fluid line 60 links the upper portion 34 (e.g., gas or vapor space) of the condensate tank 30 to the deodorization tank 40. In operation, output (e.g., steam and air) from the autoclave 20 flows through the fluid line 50 and enters into the condensate tank which can be filled with a condensing liquid such as water (e.g., lower portion 31). Thereafter such output bubbles through the condensate 32 or fluid in the condensate tank 30 and into the upper portion 34 thereof. The output (air) of the upper portion of the condensate tank 30 then flows through the fluid line 60 and enters into the lower portion 41 of the deodorizing tank 40 (and then bubbles through a deodorizing solution 42 in the deodorizer tank. This deodorizes the output and the deodorized output or deodorized air is then released from a top portion 44 or vapor space of the deodorizer tank 40 through a vent 70 such as to atmosphere.

The deodorizing solutions 42 include, for example, limonene, a colorless liquid that possesses a strong smell of oranges. The deodorizing solutions can be any such solution as known in the art or hereinafter developed that is otherwise appropriate for the intended us. The condensate is any of a known of liquids or solutions as are known to those skilled in the art and in an exemplary embodiment, the condensate is water.

In further embodiments, one or both of the condensate and deodorizing tanks are further configured with plumbing so as to allow a user to selectively and controllably remove and/or add condensate or deodorizing fluid to the respective tank.

In further embodiments or aspects of the present invention such a system can further include valves or mechanisms that can control the flow of fluid between the above identified tanks and autoclave as well as facilitating the flow of fluids between the autoclave and the above identified tanks. In yet further embodiments, the autoclave is arranged so it is elevated with respect to the condensate tank or the fluid line 50 between the autoclave and the condensate tank is configured so as to include a back-flow preventing mechanism (e.g., check valve) to preclude condensate from flowing into the autoclave.

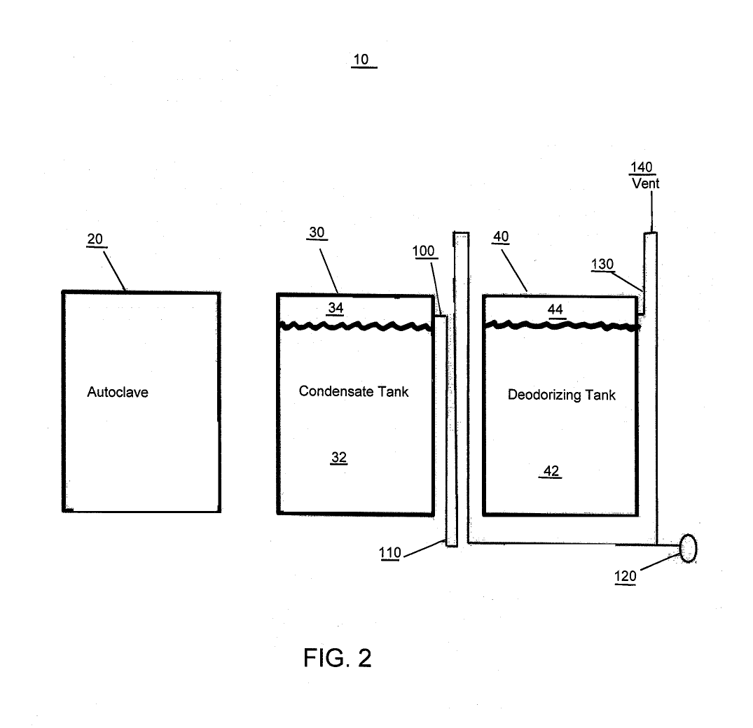

As shown in FIG. 2, the system 10 for deodorization of reclaimed steam from a waste treatment autoclave can further include overflow plumbing or piping. It should be recognized that such overflow piping or plumbing is in addition to that shown in FIG. 1. More specifically, the condensate tank 30 can further include an overflow line 100 that is fluidly coupled to the upper portion 34 of the condensate tank. In one example, the overflow line 100 is configured with a U shaped trap 110 that contains liquid therein to provide resistance to the flow of air and steam through the drain line 100. In other embodiments, the overflow line is configured or includes alternate means as are known to those skilled in the art than such a trap that can be employed to assure that steam and air follow the fluid path as described above in FIG. 1. Liquid, steam and/or air from the upper portion 23 of the condensate tank 30 that overflows can flow through the overflow line 100 to a drain or waste tank 120.

The deodorizer tank 40 can further include an overflow line 130 that is fluidly coupled to the upper portion 34 space of the deodorizer tank. Liquid, steam and/or air from the deodorizer tank 40 that overflows can flow through the overflow line 130 to the drain or waste tank 120. Such an overflow line 130 is configurable as described above for the condensate overflow line 120 to bias flow of the deodorized fluid or air to the vent.

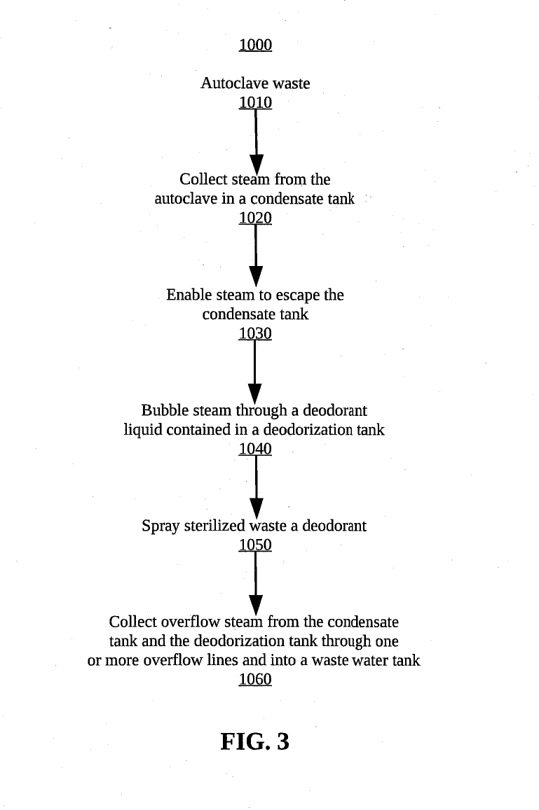

Referring now to FIG. 3, there is shown a process 1000 to deodorize reclaimed steam from a waste treatment autoclave 20 includes autoclaving (Step 1010) medical waste. Such autoclaving includes disposing the medical waste in the autoclave 20 and thereafter operating the autoclave at the appropriate operational conditions to treat the medical waste.

Such a process includes collecting (Step 1020) steam from the autoclave in a condensate tank 30 and enables (Step 1030) the steam to escape the condensate tank and bubble (Step 1040) through a deodorant liquid 42 contained in a deodorization tank 40.

In further embodiments, sterilized waste from the autoclave is sprayed (Step 1050) with a deodorant.

In yet a further embodiment, such a process includes collecting (Step 1060) overflow liquid from the condensate tank and the deodorization tank through one or more overflow lines and direct it into a waste tank 120.

While this invention has been particularly shown and described with references to preferred embodiments thereof, it will be understood by those skilled in the art that various changes in form and details may be made therein without departing from the spirit and scope of the present application as defined by the appended claims. Such variations are intended to be covered by the scope of this present application. As such, the foregoing description of embodiments of the present application is not intended to be limiting.

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A system comprising:
an autoclave for sterilization of waste, the autoclave fluidly linked to a bottom portion of a condensate tank to enable flow of steam from the autoclave to the condensate tank; and
a deodorization tank containing a liquid deodorant, an upper portion of the condensate tank fluidly linked to a bottom portion of the deodorization tank to enable steam from the condensate tank to bubble through the liquid deodorant and exit from a vent located on a top of the deodorization tank.

2. The system of claim 1 further comprising:
a waste water tank with a liquid level control system that permits liquid overflow, but in so doing resists passage of odorous steam from any path but through the deodorizer.

3. The system of claim 1 wherein the autoclave further comprises a deodorant device that sprays the sterilized waste.

4. The system of claim 1 wherein the waste is medical waste.

5. The system of claim 4 wherein the medical waste is a regulated medical waste.

6. The system of claim 4 wherein the medical waste is selected from the group consisting of gloves, needles, lancets, syringes, broken glass, scalpels, culture slides, culture dishes, broken capillary tubes, broken rigid plastic, exposed ends of dental wires, stents, laboratory slides and cover slips contaminated with infectious agents.

7. A method comprising:
sterilizing waste in an autoclave;
collecting steam from the autoclave in a condensate tank; and
bubbling steam from the condensate tank through a deodorant liquid contained in a deodorization tank.

8. The method of claim 7 further comprising:
collecting overflow from the condensate tank and the deodorization tank in a waste water tank or a drain.

9. The method of claim 7 wherein sterilizing the waste further comprises:
collecting the sterilized waste; and
spraying the collected sterilized waste with a deodorant.

10. The method of claim 7 wherein the waste is medical waste.

11. The method of claim 10 wherein the medical waste is selected from the group consisting of gloves, needles, lancets, syringes, broken glass, scalpels, culture slides, culture dishes, broken capillary tubes, broken rigid plastic, exposed ends of dental wires, stents, laboratory slides and cover slips contaminated with infectious agents.

12. The system of claim 1 wherein the liquid deodorant is limonene.

13. The method of claim 7 wherein the liquid deodorant is limonene.
